(19) United States
(12) Patent Application Publication
(10) Pub. No.: US 2011/0154729 A1
(43) Pub. Date: Jun. 30, 2011

(54) Growth media and a method of growing fungi of enhanced flavour and shelf life

(76) Inventor: Colin Sturgeon, (US)

(21) Appl. No.: 12/647,321

(22) Filed: Dec. 24, 2009

(51) Int. Cl. A01G 1/04; C12P 1/00

(52) U.S. Cl. 47/1.1; 435/41

(57) ABSTRACT

A method of composting “green waste” to provide growth media, particularly for growing fungi of enhanced flavour and shelf life, is described. The growth medium comprises a graded mulch of “green waste” comprised essentially of grass and hedge cuttings, however, wood chippings, bark, leaves and vegetable and fruit materials may also be used, together with a density modifier, such as straw, and a nitrogen additive. The growth medium is regulated to enhance growth and availability of water and nutrients during the growth of spawn into harvestable fungi. The growth medium may be adapted for use as a mushroom spawn carrier and as casing material. Additionally, a compost material suitable for domestic and more general horticultural use is provided. A soil cleanser and/or improver is also provided.

### FIELD OF THE INVENTION

The present invention relates to growth media for fungi and to a method of growing fungi of enhanced flavour and shelf life. The invention is specifically directed to growth media for and a method of growing mushrooms. More particularly, the invention relates to the commercial cultivation and growth of mushrooms having texture and flavour akin to that of wild mushrooms.

It will be appreciated by the skilled addressee that commercial/high yield mushroom growing techniques currently used either result in mushrooms having little real flavour or are cultivated using techniques now losing popularity with consumers due to their demand for more ecologically and hygienically sound practices. It is also known that commercially grown mushrooms tend to have a relatively short shelf life, resulting in high spoilage rates at point of sale.

It will further be appreciated by the skilled addressee that the term ‘mushroom’ as used herein is directed particularly to edible fungi of all types but commonly referred to by the consuming public by that term, in addition to the more specialised fungi or “exotics” appreciated by the connoisseur. The invention is not intended to be limited to edible fungi, as there exists an increasing market for fungi of other varieties for a range of industrial, agricultural and horticultural uses.

### BACKGROUND TO THE INVENTION

It is well appreciated that there are successive steps required for the production of mushrooms on a reliable commercial basis. Broadly, the growth medium must be prepared to provide an appropriate environment and seeded with mushroom mycelia; then the growth medium is cased with a wetted soil on which the mushrooms grow and must be maintained within the correct range of temperature, humidity and CO₂ concentrations.

Examining the steps in more detail, there is generally recognised within the art that there are six phases of mushroom production from raw material to harvest or cropping. Phase I involves the initial part of the composting process where the constituent materials are brought together in the desired proportions and mixed into a substantially homogeneous whole. Water is added to the compost (without over-wetting) to maintain a high moisture content and the compost is rotated regularly. During Phase II, further composting takes place to substantially eliminate any potentially harmful bacteria within the constituent materials and to reduce the levels of ammonia (NH₄) present in the compost. Subsequently, in Phase III the compost or growth medium is inoculated with mushroom mycelia of the desired mushroom strain. Although other methods can be used to introduce mushroom into the growth media, “spawning” is the most commonly used and reliable method. As the “spawn” grows it permeates the growth medium, however, before there can be further development in mushroom growth, a casing material is applied as water retaining cover to the growth medium. The application of the casing material, which can be selected from a number of different sources, comprises Phase IV. As the mushroom mycelia develop, pins form within the casing material which acts as a reservoir to ensure the mushrooms are provided with sufficient moisture to form mushrooms of the appropriate size and constitution. This “pinning” comprises Phase V where the mushroom mycelium grows towards the formation of the mushroom fruit. The development of the pins and eventual fruit is usually controlled by the amount of water available in the compost and the temperature, humidity and carbon dioxide (CO₂) concentrations of the ambient air. The final part of the process is Phase VI which is generally referred to as “cropping”. This involves the collection of flushes or breaks of mushrooms for harvest. Once collected, further flushes of mushrooms may occur until either the nutrient base is depleted or the conditions are no longer optimum for continued growth. Normally, the first flushes can be cropped 18 to 21 days from casing (Phase IV).

Looking in more details at the Phase I and II stages, the growth medium is normally a compost of horse manure, straw, chicken litter, water gypsum and nitrogen additive in various proportions of which certain ones may be replaced with a range of well-known substitute constituents. For such conventional mushroom compost, the materials are held for 7 days in a pre-wet condition, being moved every 24 hours, constantly adding water to build up moisture levels therein. Often this mixture is set out in rows for convenient adding of water and for turning every 48 hours or thereabouts. Ideally, the moisture content is built in this way until the levels reach 75-76%. Before being placed in a pasteurisation tunnel, the mushroom compost should reach a temperature of 70° C., nitrogen levels of 1.8 mg/kg and pH7 (neutral). During conditioning over 6 to 7 days in the pasteurising tunnel, temperatures within the compost reach 57° C. and the ammonia levels reach 300 ppm.

Due to the impact of numerous food scares, the food industry generally has been examining constituent components in the food chain and attempting to eliminate potential sources of bacterial and viral contamination. Traditionally, composting has been seen as a convenient way to utilise nutrients present in agricultural waste or spoils, including animal excreta and rotted foodstuffs.

As a result of increasingly fastidious consumer demands, the majority of crop growers have been examining how to ensure consumer safety against a background of increased demand and stiffer competition.

One solution to the use of animal waste, usually in the form of a manure and straw or hay mix, is to use whole or crushed grain as the primary nutrient substitute. Most suitable grains, however, have their own commercial value and consequently their use adds to the fundamental cost of production. Processing the grain as is suggested in U.S. Pat. No. 3,828,470, for example, where the growth medium comprises finely ground and conditioned cereal grains, only further adds to the production costs.

It is also known to use materials such as wood bark as a nutrient base, however, this is usually reliant on an active local timber industry and, again, wood bark has a separate valuable commercial life, for example, as a decorative garden material and harrier to weed growth, amongst other uses.

There exists an increasing and as yet untapped resource of suitable material for making compost suitable for growing fungi. This resource has been generated by the increased ecological pressure against the ecologically unsound practice of using landfill sites for domestic (and commercial) refuse. Increasingly, urban and suburban authorities have been instigating collection for selected types of recyclable waste to cut down on material destined for landfill. One of the types of material now excluded from all such refuse collections is grass cuttings and hedge trimmings, collectively referred to herein as “green waste”.

It has been found through experimentation that the mere substitution of “green waste” compost or growth media does not achieve the desired results due to insufficient levels of temperature, ammonia and nitrogen to ensure growth.

It is known that the use of different growth media, casing material and selective additives, together with adjusting methods of nurturing mushroom growth progression, has a profound effect on cropping yields and mushroom sizes. What has not been of significant concern to date is the resulting lack of flavour and shortened shelf life of mass-produced, intensively grown mushrooms. Unfortunately, to satisfy consumer demand for product, growers have concentrated on mushrooms of smaller cap size and quicker growth. Significantly, these mushrooms lack the sophisticated flavours of a more mature mushroom and they tend not to have sufficiently long shelf lives. Consequently, if the route to market is slow or if the market does not have a relatively rapid turnaround, such “immature” mushrooms will be spoiled. Another factor in a lack of developed taste is the repeated use or overuse of a given batch of growth media or casing material. Thus, in an attempt to maximise use of raw materials and to decrease production costs, flavour and longevity of the product suffers.

By modifying the nutrient base and carefully controlling the growth progression and ambient conditions, mushrooms of enhanced flavour and shelf life can be grown.

As noted above, the time period from preparation of growth media to collecting a first harvest of fungi can be from 40 days using conventional composting where spawn runs are in the order of 15 days.

It is an object of the present invention to provide compost or growth media for fungi, particularly edible mushrooms, utilising an ecologically viable “waste” product and to avoid the use of animal waste in doing so. It is also an object of the invention to provide a casing material made predominantly from non-depleting resources.

It is an object of the invention to provide a method of providing a growth medium, particularly a mushroom compost, having a lower bacterial count, improved biological properties and a shortened preparation time.

It is further an object of the present invention to provide a method of growing fungi of enhanced flavour and longer shelf life. Furthermore, the invention seeks to provide a commercially grown mushroom having a flavour akin to wild field or woodland mushrooms.

### SUMMARY OF THE INVENTION

Accordingly, the present invention provides a method of composting “green waste” to provide growth media for growing fungi of enhanced flavour and shelf life, the method comprising:
forming a mulch of screened “green waste” consisting essentially of grass and hedge cuttings and adding to this 2% to 5% dry weight of a density modifier and optionally a nitrogen additive;
pre-wetting the mulch mixture for a period of 20 to 100 hours;
wetting, blending and rotating the mulch mixture for a period of 3 to 4 days;
arranging the mulch mixture in rows or stacks and turning it every 24 hours for a period of 4 to 5 days;
adding further water to obtain a moisture level of approximately 75%; and
pasteurising the mixture.

Accordingly to a further aspect, the present invention provides a growth media for fungi, the media comprising a mulch of “green waste”, consisting essentially of grass and hedge cuttings, a density modifier, and a nitrogen additive.

The primary advantage of the growth media is that it reduces significantly the time necessary to prepare a growth medium having the desired characteristics of nutrient base, nitrogen levels and growth progression for cultivation of fungi. A further advantage is that the growth medium completely avoids the use of animal by-products, particularly excreta.

The mulch of “green waste” optionally includes materials from trees, such as wood chippings, sawdust, bark and leaves. Furthermore, the “green waste” may include waste vegetable or fruit from that deemed unfit for display (as being past a ‘sell by’ date) from supermarkets and the like to potato peelings, available in vast quantities from a variety of sources.

Advantageously, the density modifier comprises straw. Ideally, the straw comprises between 1 and 5% dry weight of the growth media.

The density modifier is used to account for the variation in density of the mulch of “green waste” which varies from season to season in accordance with the growth cycles of the available constituents. For example, grass cuttings are less likely to be form a large component of the “green waste” during winter.

The density modifier ensures that oxygen can flow within the growth media during composting.

Conveniently, the mulch is screened to control the length of raw “green waste” forming the growth medium. This allows an open structure of media to be achieved.

In a preferred embodiment, growth media in accordance with the invention comprises a mulch of “green waste”, 3% dry weight density modifier and 0.35% nitrogen additive, to which is added sufficient water to thoroughly wet the admixture.

A primary advantage of the growth medium is the reduction in composting time from 15 days to 8 days.

The invention further provides a growth medium for enhancing growth and availability of water and nutrients during the growth progression of spawn into harvestable fungi, the growth medium comprising a screened mulch of “green waste”, a density modifier, a nitrogen additive and a casing material, the growth medium being capable of retaining a substantially evenly distributed reservoir of moisture therewithin.

The present invention further provides a method of growing fungi of enhanced flavour and shelf life comprises:
forming a bed of growth media formed from a mulch of “green waste” which includes an inoculum of the desired species of fungi;
overlaying the bed of growth media with a casing material having suitable moisture retaining properties;
elevating the ambient temperature and CO₂ levels to that known to be optimum for the species of the inoculum; and
harvesting the fungi,
   characterised in that the mulch precludes the use of animal excreta and like materials thereby avoiding substantial contamination with bacteriological agents, whereby the fungi produced have increased shelf life and coincidentally enhanced flavour characteristics.

The invention further provides a mushroom spawn and a method making same wherein the growth medium is prepared using graded “green waste” with a density modifier, the growth medium providing a source of nutrition for the mycelia and the density modifier ensures the structure of the medium is such to ensure adequate oxygenation.

The above arrangement provides faster mycelial growth and increased mushroom yield.

The present invention further provides a casing material comprising a finely graded mulch of “green waste” and peat in a ratio of between 4:1 and 2:3 (80:20 percent dry weight to 40:60 percent dry weight), having a pH modifier to control the pH to between 6.6 and 7.8 (a pH of in the region of 8.8 when taken as a 1:5 dry extract is considered acceptable) and added water to adjust the moisture content to 74 to 76%.

According to a yet further aspect of the invention there is provided a growth medium for fungi comprising a screened mulch of “green waste” and a density modifier, the growth medium having a composition (measured as on a dry matter basis) including ammonium nitrogen present in the order of 12 mg/kg (12 ppm), nitrate nitrogen present in the order of 106 mg/kg (106 ppm), phosphorous present in the order of 1732 mg/kg, potassium in the order of 8753 mg/kg, magnesium in the order of 1623 mg/kg, sodium in the order of 1010 mg/kg, calcium in the order of 22958 mg/kg, sulphur in the order of 1655 mg/kg, iron in the order of 11006 mg/kg, manganese in the order of 205 mg/kg, and wherein the medium has a bulk density of 350 g/l, a water holding capacity in the range of 50-76%.

It will be appreciated by the skilled addressee that the growth media of the present invention may be provided with spawn or mycelia in situ.

It will further be appreciated that the amount of certain additives, for example, nitrogen additives, Sporavite™ and various growth enhancers may be reduced without affecting overall growth characteristics.

The invention also provides a compost material suitable for domestic and more general horticultural use, the compost material comprising a “green waste” mulch as described hereinabove.

More particularly, there is provided a soil cleanser and/or improver comprising a “green waste” mulch as described hereinabove.

### DETAILED DESCRIPTION OF THE EMBODIMENTS

The present invention will now be described more particularly with reference to the tables below which show, by way of example only, test results for the exemplifying embodiments of the invention.

Conventional growth media for mushrooms, referred to more generally as mushroom compost, comprises a mixture of the following components to a greater or lesser degree:

Straw Hay Horse manure Sporavite™
Chicken manure Rape Gypsum

It will be appreciated that one or more of the above components may be omitted without altering the overall characteristic of a conventional compost. Usually, a compost window of 15 to 21 days is required with a “moisture at fill” level of 76%. The second composting phase normally takes between 4 and 6 days.

Mushroom growth media is made from screened “green waste” comprising domestic grass and hedge cuttings together with a relatively small proportion of wood product, for example, tree prunings. The “green waste” is mulched or otherwise sized and screened to give an approximate guide to the length of constituents of the resultant mulch. Where the structure of the growth media is likely to be too tight, that is, dense, straw is added as a density modifier so that when the media is used, the mycelia or spawn therein. Thus, using screened “green waste” with 3% by weight of straw, together with an inoculum such as Sporavite, an improved growth media is provided. The compost window is significantly reduced to just 8 days and the “moisture at fill remains at 76%. The second composing phase remains between 4 and 6 days.

To demonstrate the advantages of the growth media of the present invention, it is useful to examine a number of characteristics of conventional compost and compare this with the characteristics of the “green waste” mushroom compost of the present invention. With conventional compost, a case run temperature in the range of 21° C. to 23° C. is to be expected. During days 0 to 4 water is present in the case run and there is water available at the end of the “flush” for the next “flush”. Furthermore, a fluctuation in air temperature at the end of the “flush” is noted. With the “green” mushroom compost or growth invention of the present invention, the case run temperature is between 21° C. and 26° C. Water is present in the case run between days 2 and 5 and no water is available at the end of the “flush”. Further notable characteristics using the “green” compost include the presence of extra water at the start of the “flush” and misting during the “air up” process. Higher CO₂ levels are noted and consequently closer control of the ambient air is required.

Table 1 below shows laboratory test results for the growth media made using “green waste” as described. The analysis method used is as recommended by the Composting Association. Figures shown in parenthesis adjacent a result are the maximum limits quoted for potentially toxic elements. The limits are set by the Composting Association. In the test result shown in Table 1, the presence of these potentially toxic elements is low. It will be appreciated by the skilled reader that the tests were conducted on a “sample as received” basis and, following extraction of water, on a “dry matter” basis to more accurately evaluate concentration of the tested elements and compounds. The test was conducted by Gooch Gosforth Ltd, Analytical & Consulting Chemists, of Ipswich Road, Needham Market, Suffolk, IP6 8EL, United Kingdom under report reference number GGL2078.

TABLE 1
LABORATORY TEST REPORT FOR GROWTH MEDIA

| Parameter | Units | Result |
|---|---|---|
| Lab Bulk Density | g/l | 350 |
| Loss at 105° C. taken to be moisture | % m/m | 51.5 |
| Dry matter content, from loss at 105° C. | % m/m | 48.5 |
| pH of 1:5 extract | | 8.8 |
| Electrical Conductivity of 1:5 extract | μS/cm | 520 |
| | mS/m | 52 |
| Extractable in water - sample as received basis | | |
| NH₄ - Nitrogen as N | mg/litre | 2 |
| NH₄ - Nitrogen as N | mg/litre | 18 |
| Total extractable- sample as received basis | | |
| Phosphorous as P | mg/litre | 294 |
| Potassium as K | mg/litre | 1486 |
| Magnesium as Mg | mg/litre | 276 |
| Sodium as Na | mg/litre | 171 |
| Calcium as Ca | mg/litre | 3897 |
| Sulphur as S | mg/litre | 281 |
| Iron as Fe | mg/litre | 1868 |
| Manganese as Mn | mg/litre | 35 |
| Boron as B | mg/litre | 4.2 |
| Molybdenum as Mo | mg/litre | 0.4 |
| Copper as Cu | mg/litre | 4.2 |
| Zinc as Zn | mg/litre | 18 |
| Cadmium as Cd | mg/litre | 0.1 |
| Chromium as Cr | mg/litre | 2.4 |
| Nickel as Ni | mg/litre | 2.0 |
| Lead as Pb | mg/litre | 8.8 |
| Mercury as Hg | mg/litre | 0.02 |
| Extractable in water - dry matter basis | | |
| NH₄ - Nitrogen as N | mg/kg | 12 |
| NH₄ - Nitrogen as N | mg/kg | 106 |
| Total extractable- dry matter basis | | |
| Phosphorous as P | mg/kg | 1732 |
| Potassium as K | mg/kg | 8753 |
| Magnesium as Mg | mg/kg | 1623 |
| Sodium as Na | mg/kg | 1010 |
| Calcium as Ca | mg/kg | 22958 |
| Sulphur as S | mg/kg | 1655 |
| Iron as Fe | mg/kg | 11006 |
| Manganese as Mn | mg/kg | 205 |
| Boron as B | mg/kg | 25 |
| Molybdenum as Mo | mg/kg | 2.4 |
| Copper as Cu | mg/kg | 25* (200) |
| Zinc as Zn | mg/kg | 106* (400) |
| Cadmium as Cd | mg/kg | 0.4* (1.5) |
| Chromium as Cr | mg/kg | 14* (100) |
| Nickel as Ni | mg/kg | 12* (50) |
| Lead as Pb | mg/kg | 52* (150) |
| Mercury as Hg | mg/kg | 0.1* (1.0) |

*Low result - max for compost shown in parentheses
Report ref: GGL2078

Table 2 is a microbiological analysis of the growth media in accordance with the present invention again conducted by Gooch Gosforth Ltd, under report reference number GGL1966. The results as interpreted by a qualified microbiologist indicate that the results are very good and show that the material has undergone an effective composting treatment.

It is of particular note that the number of colony forming units (cfu) is particularly low with respect to media/mushroom compost made using horse manure, chicken droppings or other animal waste. It is of further note that these results are achieved in a shortened composting period and facilitate the growth of fungi having excellent flavour and shelf life.

TABLE 2
MICROBIOLOGICAL ANALYSIS OF GROWTH MEDIA

| Bacteria | Units | Result |
|---|---|---|
| E. Coli | cfu/gram | <100 |
| Salmonella SP. | per 25 grams | not detected |
| Presumptive | cfu/gram | 180 |
| Enterobacteriaceae | | |
| Presumptive | cfu/gram | 9400 |
| Clostridium Species | | |

cfu = colony forming units
Report Ref: GGL1966

Table 3 is a microbiological analysis of the growth media prepared in accordance with the method of the invention as conducted by Direct Laboratory Services Ltd, of Woodthorne, Wergs Road, Wolverhampton, WV6 8TO, United Kingdom. The results accorded a laboratory Reference No. 23020738, indicate that no mycotoxins were found in the tested sample.

TABLE 3
MICROBIOLOGICAL ANALYSIS OF GROWTH MEDIA

| Mycotoxin | Result | Detection Limit | |
|---|---|---|---|
| Aflatoxin B1, B2, G1, G2 | Not detected | λ | ppb |
| Zearalenone (F2) | Not detected | 50 | ppb |
| T2 Toxin | Not detected | 50 | ppb |
| Vomitoxin (DON) | Not detected | 200 | ppb |
| Ochratoxin | Not detected | 5 | ppb |

Lab ref: 23020738

After composting, the growth medium is inoculated with the required type or strain of mushroom. These can be selected from any of the white and off-white varieties of Agaricus biporus, Morchella sp. (morel); Pleurotus sp. (oyster); Lentinus edodes (shiitake); Flammulina velutipes; and Volvariella volvacea. As stated above, the inoculum is usually mycelia from which, under the appropriate conditions of temperature, humidity and oxygenating air, mushroom spawn evolves. The mushroom growth progression continues after the spawn-rum growth medium is covered in a casing material which can be selected from traditional peat based mixes, new or recycled compost, clay or the like or a mix comprising a more finely screened or graded mulch of “green waste”, together with such additives required to adjust pH and assure the casing material acts as a suitable moisture reservoir.

Table 4 is a microbiological analysis of the mushroom grown in the “green waste” growth media of the invention, conducted by Direct Laboratory Services Ltd under Laboratory Reference No. 23023723. The results indicate very good results with a mushroom of high microbiological quality.

TABLE 4
MICROBIOLOGICAL ANALYSIS OF MUSHROOMS

| Determination | Result | Units | Method ref. |
|---|---|---|---|
| Escherichia coli 44° C. | <10 | cfu/g | Micro/027 |
| Enterobacteriaceae 37° C. | <10 | cfu/g | Micro/007 |
| Listeria spp. | Not detected | /25 g | Micro/013 |
| Pseudemonads | 2900 | cfu/g | Micro/121 |
| Salmonella spp. 37° C. | Not detected | /25 g | Micro/009 |
| Total viable count 30° C. | 14 | cfu/g | Micro/001 |

Lab ref: 23023723

It has been found that mushrooms of a number of varieties or strains grown in growth media made in accordance with the invention have a higher dry weights similar to wild mushrooms and take on a more “natural” appearance with an enforced shelf life.

The result tables above show that “green waste”, when used with other substantially inert materials, produces a compost which is very low in potentially harmful bacteria. By extracting the use of materials which before composting and pasteurisation harbour bacteria known to be harmful, low counts of such bacteria are found. As a consequence, food safety regulations are easier to comply with, a ready resource is exploited, and the production is more environmentally friendly, with reduced costs and satisfactory production yields.

With the noted production yields and faster turnaround from Phase I to Phase II, an overall reduction in production cost results.

It will be appreciated by the skilled addressee that a casing material may be formed using a “green waste” compost mixed with traditional peat or clay based casing materials.

It will of course be understood that the invention is not limited to the specific details described herein, which are given by way of example only, and that various modifications and alterations are possible within the scope of the present invention.

1. A method of composting “green waste” to provide growth media for growing fungi of enhanced flavour and shelf life, the method comprising:
forming a mulch of screened “green waste” consisting essentially of grass and hedge cuttings and adding to this 2% to 5% dry weight of a density modifier and optionally a nitrogen additive;
pre-wetting the mulch mixture for a period of 20 to 100 hours;
wetting, blending and rotating the mulch mixture for a period of 3 to 4 days;
arranging the mulch mixture in rows or stacks and turning it every 24 hours for a period of 4 to 5 days;
adding further water to obtain a moisture level of approximately 75%; and
pasteurising the mixture.

2. Growth media for fungi, the media comprising a mulch of “green waste”, consisting essentially of grass and hedge cuttings, a density modifier, and a nitrogen additive, whereby the growth media reduces significantly the time necessary to prepare a growth medium having the desired characteristics of nutrient base, nitrogen levels and growth progression for cultivation of fungi.

3. Growth media as claimed in claim 2, in which the growth medium completely avoids the use of animal by-products, particularly excreta.

4. Growth media as claimed in claim 2, in which the mulch of “green waste” includes materials from trees, such as wood chippings, sawdust, bark and leaves.

5. Growth media as claimed in any one of the claims 2, in which the “green waste” includes materials ranging from waste vegetable or fruit from that deemed unfit for display (as being past a ‘sell by’ date) from supermarkets and the like to potato peelings, available in vast quantities from a variety of sources.

6. Growth media as claimed in any one of the claims 2, in which the density modifier comprises straw.

7. Growth media as claimed in claim 2, in which the density modifier comprises between 1 and 5% dry weight of the growth media.

8. Growth media as claimed in any one of the claims 2, in which the density modifier is used to account for the variation in density of the mulch of “green waste” which varies from season to season in accordance with the growth cycles of the available constituents and to ensure that oxygen can flow within the growth media during composting.

9. Growth media as claimed in any one of the claims 2, in which the mulch is screened to control the length of raw “green waste” forming the growth medium, thereby regulating the open structure of media produced.

10. Growth media as claimed in any one of the claims 2, in which the media comprises a mulch of “green waste”, 3% dry weight density modifier and 0.35% nitrogen additive, to which is added sufficient water to thoroughly wet the admixture.

11. Growth media as claimed in any one of the claims 2, in which the composting time of the growth medium is the reduced from 15 days to 8 days.

12. A growth medium for enhancing growth and availability of water and nutrients during the growth progression of spawn into harvestable fungi, the growth medium comprising a screened mulch of “green waste”, a density modifier, a nitrogen additive and a casing material, the growth medium being capable of retaining a substantially evenly distributed reservoir of moisture therewithin.

13. A method of growing fungi of enhanced flavour and shelf life comprises:
forming a bed of growth media formed from a mulch of “green waste” which includes an inoculum of the desired species of fungi;
overlaying the bed of growth media with a casing material having suitable moisture retaining properties;
elevating the ambient temperature and CO₂ levels to that known to be optimum for the species of the inoculum; and
harvesting the fungi,
characterised in that the mulch precludes the use of animal excreta and like materials thereby avoiding substantial contamination with bacteriological agents, whereby the fungi produced have increased shelf life and coincidentally enhanced flavour characteristics.

14. Mushroom spawn comprising growth media formed from a mulch of “green waste” and an inoculum of the desired species of fungi, wherein the growth medium is prepared using graded “green waste” with a density modifier, the growth medium providing a source of nutrition for the mycelia and the density modifier ensures the structure of the medium is such to ensure adequate oxygenation, whereby faster mycelial growth and increased mushroom yield is realised.

15. A method of making a mushroom spawn comprising a growth medium and an inoculum of the desired species of fungi or already-formed mycelia wherein the growth medium is prepared using graded “green waste” with a density modifier, the growth medium providing a source of nutrition for the mycelia and the density modifier ensures the structure of the medium is such to ensure adequate oxygenation, whereby faster mycelial growth and increased mushroom yield is realised.

16. A casing material comprising a finely graded mulch of “green waste” and peat in a ratio of between 4:1 and 2:3 (80:20 percent dry weight to 40:60 percent dry weight), having a pH modifier to control the pH to between 6.6 and 7.8 (a pH of in the region of 8.8 when taken as a 1:5 dry extract is considered acceptable) and added water to adjust the moisture content to 74 to 76%.

17. A growth medium for fungi comprising a screened mulch of “green waste” and a density modifier, the growth medium having a composition (measured as on a dry matter basis) including ammonium nitrogen present in the order of 12 mg/kg (12 ppm), nitrate nitrogen present in the order of 106 mg/kg (106 ppm), phosphorous present in the order of 1732 mg/kg, potassium in the order of 8753 mg/kg, magnesium in the order of 1623 mg/kg, sodium in the order of 1010 mg/kg, calcium in the order of 22958 mg/kg, sulphur in the order of 1655 mg/kg, iron in the order of 11006 mg/kg, manganese in the order of 205 mg/kg, and wherein the medium has a bulk density of 350 g/l, a water holding capacity in the range of 50-76%.

18. A growth medium for fungi as claimed in claim 17, in which there is provided spawn or mycelia in situ.

19. A growth medium for fungi as claimed in claim 17, in which the amount of certain additives, such as nitrogen additives, Sporavite™ and various growth enhancers may be reduced without affecting overall growth characteristics.

20. A compost material suitable for domestic and more general horticultural use, the compost material comprising a “green waste” mulch based growth media as claimed in claim 2.

21. A soil cleanser and/or improver comprising a “green waste” mulch based growth media as claimed in claims 2.
